(19) United States
(12) Patent Application Publication
(10) Pub. No.: US 2008/0269700 A1
(43) Pub. Date: Oct. 30, 2008

(54) Ostomy pouch

(76) Inventors: Lois Diane O'Toole, Carmel, IN (US); Daniel S. O'Toole, Carmel, IN (US)

Correspondence Address: BOWMAN & ASSOCIATES, 1016 3rd Avenue, SW, Suite #106, CARMEL, IN, 46032, US

(21) Appl. No.: 11/881,104

(22) Filed: Jul. 25, 2007

Related U.S. Application Data
(63) Continuation-in-part of application No. 11/789,265, filed on Apr. 24, 2007.

(51) Int. Cl. A61F 5/44

(52) U.S. Cl. 604/332

(57) ABSTRACT

An ostomy pouch is formed to conceal, obscure, hide, cloak, or otherwise prevent the viewing of contents of the ostomy pouch except for a “sight glass”, “bulls-eye”, area or window formed in the pouch and positioned therein for viewing at least a portion of a stoma receptor of the ostomy pouch. The ostomy pouch is formed by a plastic bag incorporating a stoma receptor or opening in one side of the bag and a transparent, translucent or clear window or area in another side of the bag. The transparent window is preferably, but not necessarily, positioned opposite and adjacent the stoma receptor. The ostomy pouch may be entirely sealed except for a stoma receptor orifice or may be open at one end for emptying its contents and incorporate or use a closure at the open end.

### RELATED APPLICATIONS

This patent application is a continuation-in-part of U.S. patent application Ser. No. 11/789,265 filed Apr. 24, 2007 entitled “Ostomy Pouch” and hereby claims the benefit of and/or priority thereto.

### BACKGROUND OF THE INVENTION

1. Field of the Invention

The present invention relates to ostomy appliances and, in particular, to an ostomy bag or pouch used for the collection of waste material.

2. Background Information

Persons may undergo major abdominal surgery, known as an ostomy, whose normal bowel function, bladder function and/or different parts of the gastrointestinal and urinary tract are or have become impaired due to a birth defect, injury, disease or other disorder. Ostomy surgery often involves removal or bypass of impaired or diseased tissue and the creation of a hole in the abdominal wall of the patient through which a section of the bowel or bladder extends. The portion of the protruding tissue (e.g. the intestine or bladder) is referred to as a stoma or abdominal stoma. The three most common types of abdominal stoma result from and may be classified as a colostomy, ileostomy or urostomy (“ostomies”). Bowel attachment surgery is termed a ileostomy in which the small intestine is involved and is termed a colostomy when the large intestine is involved. A urostomy involves the bladder.

In all ostomies the patient is unable to control the passage of bodily waste material from the stoma and thus involuntarily discharges such waste material from the stoma. Because of this, the waste material is collected by an ostomy pouch that is positioned over the stoma and attached to the user's abdomen. Users whose discharged is of a solid consistency usually employ a disposable pouch. The term disposable means that the pouch is disposed of after a single use. Users whose discharge is of a liquid consistency usually employ a collecting pouch having a bottom opening that permits the contents to be emptied and the pouch reused for a period of time. Such pouches include some type of closure that seals the bottom opening while the pouch is collecting the waste material. Various types of closure systems have been developed and patented (see, e.g. U.S. Pat. No. 4,403,991 and the discussion therein).

Attachment of the ostomy appliance to the ostomy patient is achieved with either a one piece system or a two piece system. In the two piece system, a flange is adhered to the body of the ostomy patient proximate the stoma and a bag device for waste collection. The pouch is mechanically coupled to the flange which allows the bag to be removably fastened to the flange and be in communication with the stoma. In the one piece system, both the means of adhering the bag to the stoma and the body and the bag are integral. Current adhesive systems provide an inner layer over which a protective paper or cloth tape is adhered to provide a barrier through which waste material will not pass but that allows oxygen and water vapor to pass.

In both cases, the ostomy bag or pouch is made from plastic such as polyvinyl chloride (PVC). The bags are typically formed from layers of plastic. Because ostomy bags hold human waste material, it is desirable from everyone's perspective to obscure the contents of a user's ostomy bag and/or otherwise shield its contents from view by both the user and a third party. This has heretofore been accomplished by using opaque and/or colored plastic for the pouch. A woven cotton layer may then be provided over the plastic. Since the plastic pouch is opaque or colored, neither the user nor a third party can see the contents of the pouch. Another manner of concealing the contents of the ostomy pouch and one which provides a more aesthetically pleasing appearance is ostomy bags made of opaque or colored plastic in various configurations or iconic shapes to disguise the function of the bag. This is exemplified in U.S. Patent Publication 2005/0261645 by Conrad et al., wherein ostomy bags are disclosed having an iconic shape such as a teddy bear, a purse, a heart, a butterfly, a seashell, a dog, a cat, or the like.

While the above prior art ostomy bags perform their intended function of hiding, concealing and/or obscuring their contents, they also hide, conceal and/or obscure the bag's stoma opening and/or flange assembly used to attach the ostomy bag to the user's stoma. Thus, the bag's stoma opening cannot be seen through the bag. For users that need to be able to see the bag's stoma opening in order to easily register the bag's stoma opening with the user's stoma/stoma flange during installation, this creates a problem. Since the bag's stoma opening cannot be seen through the bag, it makes it quite difficult to properly attach the bag when the ostomy bag's stoma opening cannot be seen from the reverse side as when looking in a mirror in order to attach the ostomy bag. This makes a user tend toward using the transparent ostomy bags. Thus, however, sacrifices discreetness for ease of use.

It is thus apparent from the above that here there is a need for a discreet type of ostomy bag or pouch that is also easy to attach to a user's stoma.

### SUMMARY OF THE INVENTION

The present invention is an ostomy bag, pouch or appliance formed to conceal, obscure, hide, cloak, or otherwise prevent or hinder the viewing of contents thereof and/or therein except for a transparent or semi-transparent window or area of and/or formed in the pouch and positioned for viewing at least a portion of a stoma receptor and/or a stoma opening of the stoma receptor of the ostomy pouch.

In one embodiment, the ostomy pouch is formed by an opaque plastic bag incorporating a stoma receptor in one side of the bag and a transparent window in another side of the bag. The transparent window is preferably, but not necessarily, positioned opposite and adjacent the stoma receptor. The ostomy pouch may be entirely sealed except for a stoma receptor orifice or may be open at one end for emptying its contents and incorporate or use a closure at the open end.

In another form, the ostomy pouch is formed by an opaque, non-transparent or non-translucent plastic incorporating a stoma receptor or opening in one side of the pouch and a transparent or semi-transparent window or area in another side or area of the pouch and configured to allow a user to see the stoma opening during placement. The ostomy pouch may be entirely sealed except for the stoma opening or may be open at one end for emptying its contents and incorporate or use a closure at the open end.

In another form, the ostomy pouch is formed by a clear, translucent, or transparent plastic incorporating a stoma receptor or opening in one side of the plastic pouch. An opaque, non-transparent, non-translucent or semi-opaque, semi-non-transparent, or semi-non-translucent layer is provided over at least a side of the pouch that is away from the body of the user when placed on the user with a transparent viewing window or area therein or a cutout therein. The viewing window, area or cutout is positioned to allow the user to see at least a portion of the stoma receptor or opening during placement of the ostomy pouch on the user.

In another form, the ostomy pouch is formed by a semi-clear, semi-translucent or semi-transparent plastic incorporating a stoma receptor or opening in one side of the plastic pouch. An opaque, non-transparent, non-translucent or semi-opaque, semi-non-transparent, or semi-non-translucent layer is provided over at least a side of the pouch that is away from the body of the user when placed on the user with a clear, transparent or translucent viewing window or area therein or a cutout therein. The viewing window, area or cutout is positioned to allow the user to see at least a portion of the stoma receptor or opening during placement of the ostomy pouch on the user.

The layer may be formed of plastic, cloth, paper or other material and may constitute a decal, a sticker, a label, an overlay, paint, markings, ink, a coat or coating, or a sheet, film, deposit, lamina, stratum or the like.

In another form, the ostomy pouch or at least a portion thereof is formed from a thermochromic, color-changing or transparent/translucent-to-opaque plastic and incorporates a stoma receptor or opening in one side of the pouch. In one form thereof, such plastic is initially a light transparent or translucent color, clear, translucent or transparent that allows the user to easily see the stoma receptor or opening and thus install the ostomy pouch but, which after installation, turns opaque, non-transparent, non-translucent, semi-opaque, semi-non-transparent or semi-non-translucent, or turns an opaque, non-transparent, non-translucent, semi-opaque, semi-non-transparent or semi-non-translucent color due to the user's body heat (i.e. an increase in temperature).

In another form, the ostomy pouch or at least a portion thereof is formed from a clear, transparent, translucent, semi-clear, semi-transparent, semi-translucent plastic with a thermochromic ink or paint layer, overlay, stratum, coat or coating except for a clear, transparent or translucent viewing window or area positioned to allow the user to see at least a portion of a stoma receptor or opening of the pouch to allow the user to install the ostomy pouch.

In one or more of the forms, the ostomy pouch may incorporate a date area where a user may write the day of the week or apply a day of the week sticker or decal indicating when the pouch was installed.

The various embodiments of the present ostomy pouch thus provide a discreet ostomy appliance that still allows for easy attachment to the user by giving the user a manner of viewing attachment features of the ostomy appliance side while the remainder of the appliance hides or obscures the contents of the ostomy appliance.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a plan view of a back side of an ostomy pouch fashioned in accordance with the principles of the present invention;

FIG. 2 is a plan view of a front side of the ostomy pouch of FIG. 1;

FIG. 2A is a plan view of an alternative embodiment of the front side of the ostomy pouch of FIG. 1;

FIG. 2B is a plan view of another alternative embodiment of the front side of the ostomy pouch of FIG. 1;

FIG. 3 is a sectional view of the ostomy pouch of FIG. 1 taken along line 3-3 of FIG. 2;

FIG. 4 is an enlarged top side perspective view of a stoma receptor of the ostomy pouch of FIG. 1;

FIG. 5 is an enlarged top side perspective view of an alternative embodiment of a stoma receptor for an ostomy pouch per the present principles;

FIG. 6 is an enlarged bottom side perspective view of a stoma receptor of the present ostomy pouch;

FIG. 7 is a sectional view of an alternative embodiment of an ostomy pouch fashioned in accordance with the present principles; and

FIG. 8 is a front plan view of an ostomy pouch fashioned in accordance with the present principles incorporating a day of the week area.

Like reference numerals indicate the same or similar parts throughout the several figures.

A detailed description of the features, functions and/or configuration of the components depicted in the various figures will now be presented. It should be appreciated, however, that not all of the features of the components of the figures may be necessarily described in detail. Some of these non discussed features as well as discussed features are inherent from the figures. Other non discussed features may be inherent in component geometry and/or configuration.

### DESCRIPTION OF PREFERRED EMBODIMENTS

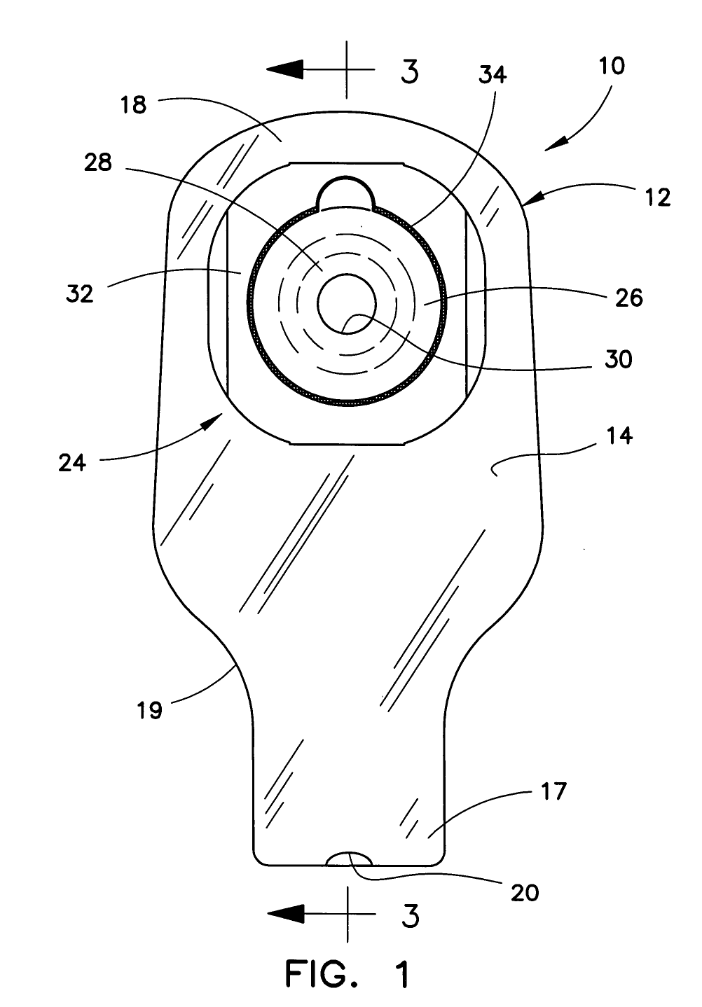

Referring to FIGS. 1, 2, 3, 4 and 5 there is depicted an exemplary embodiment of an ostomy pouch, appliance or bag (collectively, pouch), generally designated 10, fashioned in accordance with the principles of the present invention as described herein, particularly for use with a stoma of a human user and for the collection of human waste material as a result of an ostomy. The ostomy pouch 10 may be fashioned as a drainable pouch or a collection-only pouch but is preferably, but not necessarily, a disposable type ostomy pouch. The ostomy pouch 10 is formed by a preferably plastic body 12 defining a bag or pouch. The plastic may be one that is known in the art for manufacturing ostomy bags. Other plastics and/or materials, however, may be used. The body 12 is formed by a first side 14 and a second side 16 lying over, opposite of and/or adjacent to the first side 14. The first and second sides 14, 16 may be separate pieces or may be unitary. Moreover, the first and second sides 14, 16 may be formed by or of one or more layers as appropriate. The first side 14 may be considered a back side since, when installed, the pouch is adjacent the user and thus not seen when looking at the user. As such, the second side 16 may be considered a front side since, when installed, the pouch is away from the user and thus is seen when looking at the user.

In one form, the first and second sides 14 and 16 are formed of an opaque, non-transparent or non translucent plastic such that the contents of the pouch are obscured. In another form, the first and second sides 14 and 16 are formed of a semi-opaque, semi-non-transparent or semi-non-translucent plastic. In another form, the first side 14 is formed of a clear, translucent or transparent plastic while the second side 16 is formed of an opaque, non-transparent, non-translucent, semi-opaque, semi-nontransparent or semi-translucent plastic. The plastic may also be colored or otherwise marked (such as described below) so as to hide, obscure, mask, blur, or block out the contents of the pouch from at least the front side thereof. The term opaque as used herein encompasses all such synonyms, manners and/or markings.

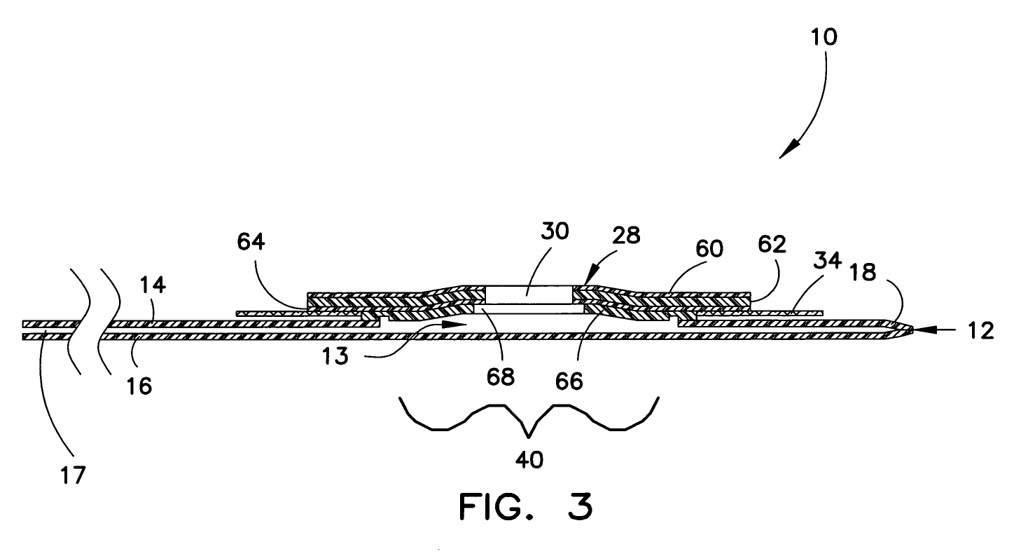

As best discerned in FIG. 3, the first and second sides 14 and 16 are sealed or joined together at one end 18 so as to form an interior 13 for the collection of waste material. The first and second sides 14 and 16 are shaped so as to form a tapered section or neck 19 that is distal the end 18. The neck 19 terminates in end 17. In the embodiment shown, the end 17 is open (i.e. the first and second sides 14, 16 are not joined or sealed) and includes a cutout 20. Since the end 17 is open, the end 17 is shaped and/or configured to receive a closure device (not shown) for temporarily closing or sealing the end opening 17. Alternatively, the end 17 may include an integral closure or closure device such as is known in the art. It should be appreciated that the sides 14 and 16 and thus the pouch 10 may take on different shapes than the one shown herein.

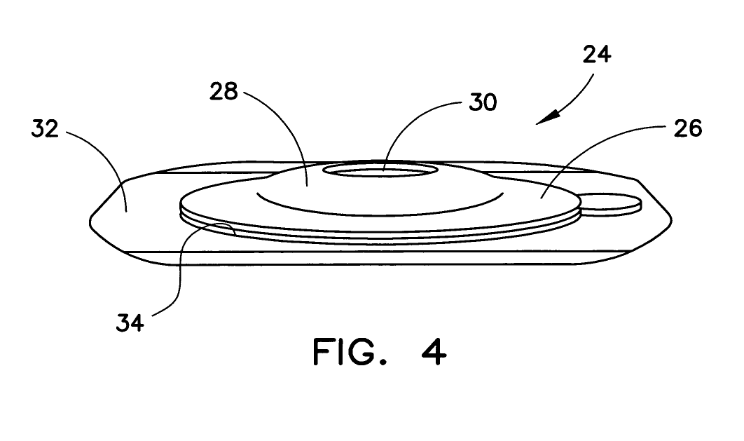

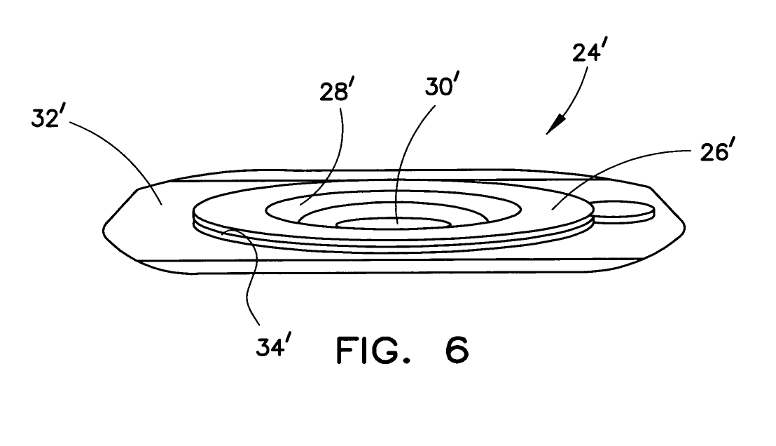

The first side 12 is the side of the pouch 10 that is positioned inwardly toward the user or next to the user's skin when the pouch is installed onto the user's stoma (not shown). As such, the first side 12 has a stoma receptor 24 that is configured to be received onto and about the user's stoma/stoma area and adhered thereto such as is known in the art. The stoma receptor 24 is the manner of attachment and communication with the user's stoma. The stoma receptor 24 includes a generally annular convex base portion 26 terminating in a conical convex nozzle 28 having an orifice or opening 30. The opening 30 is typically round. The stoma receptor 24 may thus be termed a convex stoma receptor and this is particularly seen in FIG. 4. The convex stoma receptor 24 is used on/with a corresponding stoma. It should be appreciated that the stoma receptor may take different forms. FIG. 6 depicts an alternative embodiment of a stoma receptor 24′. The stoma receptor 24′ is likewise configured to be received onto and about the user's stoma/stoma area and adhered thereto such as is known in the art. The stoma receptor 24′ includes a generally annular concave base portion 26′ terminating in a conical concave nozzle 28′ having an orifice or opening 30′. The stoma receptor 24′ may thus be termed a concave stoma receptor. The concave stoma receptor 24′ is used on/with a corresponding stoma. Other configurations are contemplated and encompassed by the term stoma receptor.

As best seen in FIG. 3, the base portion 26 is formed of multiple plies of material. Particularly, the base portion 26 has an outer covering layer 60 (e.g. paper) that is overlaid onto a cushioning/cushiony material layer 62. A layer of adhesive is provided on the outside surface of the cushion layer 62. The outer covering layer 60 is removed for use thereby exposing the adhesive layer. Below the cushion layer 62 is a transition layer 64 that may be made of a fibrous material. Below the transition layer 64 is the mesh layer 34. The mesh layer 34 extends over and is interposed between the plastic pouch 12 and adhesion layers of the stoma receptor 24. A rigid (e.g. plastic) coupling ring 66 is provided between the mesh layer 34 and the plastic pouch 12. The coupling ring 66 includes a round opening 68 corresponding to opening 30 (and may be considered as one) and provides sealing to and between the stoma receptor and the plastic pouch such that the opening 30 is in communication with the interior 13 of the pouch 12. Of course, it should be appreciated that the configuration, structure and/or materials of the stoma receptor may be different than that described and/or modified as desired. Moreover, the manner of communication, adhesion and/or coupling of the stoma receptor/pouch may be different than that described and/or modified as desired.

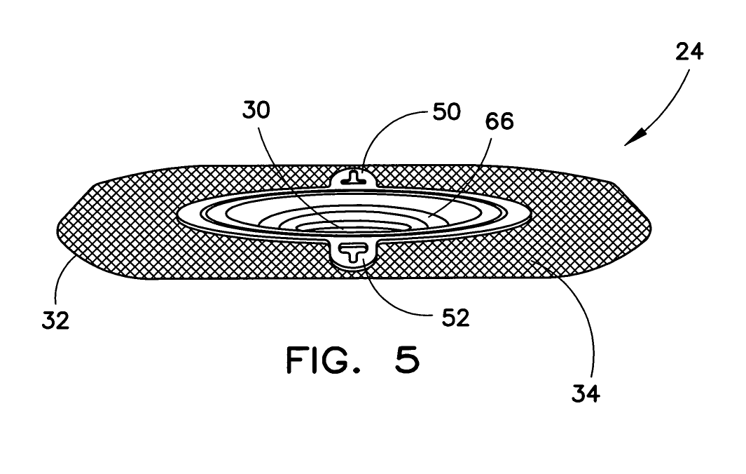

The exposed portion of the stoma receptor 24 (the portion that is adjacent to the user's skin) is particularly shown in FIG. 4. FIG. 5 shows a backside view of the stoma receptor 24. Particularly, the mesh layer 34 is shown which is between the plastic pouch 12 and the plastic coupling ring 66. The coupling ring 66 includes coupling flanges 50 and 52 that are used to attach the ostomy appliance 10 to the user. Of course, other configurations may be used.

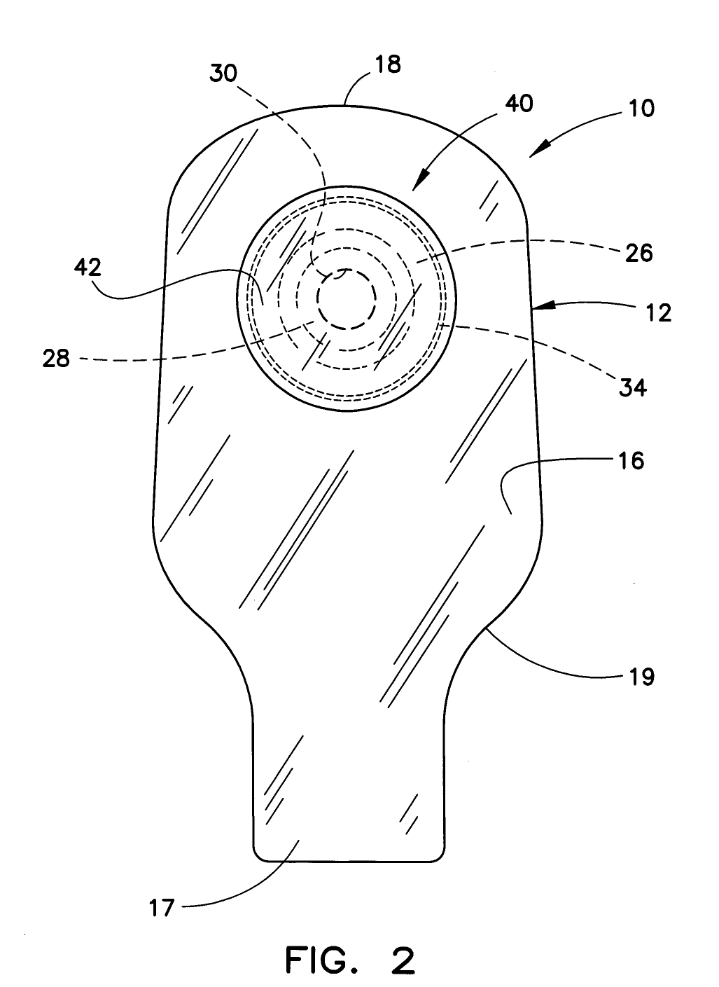

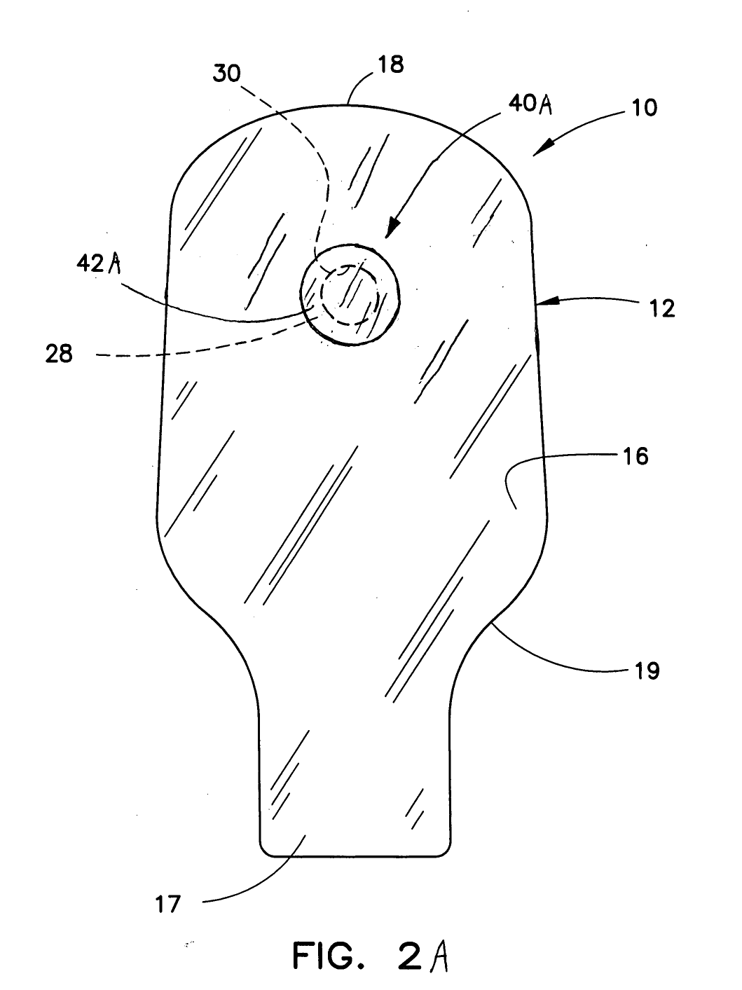

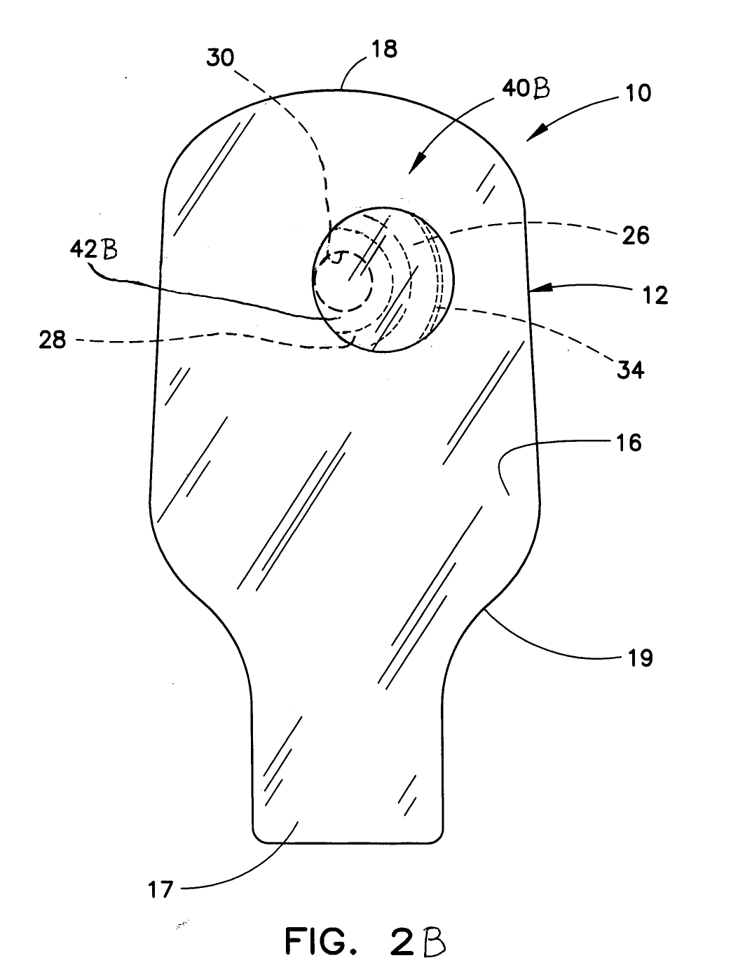

Referring now to FIG. 2, the second side 16 of the pouch 10 is shown. The second side 16 of the pouch is the side that faces outwardly from the user when installed and thus has been termed the front of the pouch. In accordance with the principles of the present invention, the second side 16 includes a window, porthole, area or the like 40 (collectively, window) that is shown situated opposite the stoma receptor 24 and particularly the nozzle 28 and preferably, but not necessarily, is formed integral with the second side 14. The window 40, however, may be formed independently of the second side 14. The window 40 is formed by a transparent, translucent, clear, semi-transparent, semi-translucent or semi-clear plastic portion 42 such that appropriate portions of the stoma receptor 24 (e.g. the nozzle and the opening 30) can be seen/viewed from the second side 16 (which may be considered the front side of the pouch when worn by a user, which makes the first side 14 the back side when worn by a user). The size and/or configuration of the window 40 may be modified as desired. As such, and without being exhaustive, the window 40 may be circular, oval, square, triangular, polygonal or otherwise. The window 40 allows the user to see the stoma opening or orifice 30 of the pouch 10 for easier attachment of the pouch 10 to the user. Since in the various forms of an ostomy pouch made in accordance with the present principles the remainder of the pouch 10 is made from an opaque plastic or one or more materials, layers, coatings, etc., the majority of the contents of the pouch 10 is obscured, hidden, masked, blurred, or blocked out from external view.

While the window 40 in FIG. 2 is shown opposite the stoma receptor 24, it should be appreciated that the window 40 may be positioned anywhere on the second side 16 such that the window allows the user to see at least a portion of the stoma receptor or the stoma opening thereof while positioning, placing or installing the pouch thereon. Moreover, the window may be positioned in the side of the pouch, the first side 14 or otherwise.

As such, reference is made to FIG. 2A which shows the ostomy pouch 10 with a window 40A that is smaller than the window 40. The window 40A is formed by a clear, translucent or transparent cover, covering, layer, lamina or the like 42A (integral or not integral with the front side thereof) that is opposite the stoma receptor 24 and particularly is directly opposite the stoma opening 30. The window 40A is sized to allow viewing of only the stoma opening or orifice 30 from the front side of the pouch. In FIG. 2B, there is shown the ostomy pouch 10 with a window 40B likewise formed by a clear, translucent or transparent cover, covering, layer, lamina or the like 42B (integral or not integral with the front side thereof) that is offset from being directly opposite the stoma receptor 24 and/or the stoma opening or orifice 30. In addition, the window 40B has a size that is between the size of the window 40 and the size of the window 40A.

It should be appreciated that the present ostomy pouch may be fashioned in other manners, shapes, perimeter shapes, iconic shapes, configurations and/or the like while incorporating a window or viewing area of preferably, but not necessarily, a transparent, translucent or clear material in the pouch for viewing the stoma receptor, the stoma opening or orifice and/or the stoma attachment features for aligning and/or proper attachment of the pouch by the user to the user (user's stoma). The window may have a pattern itself or other markings as long as they provide at least a limited view of the stoma attachment features on the front side of the pouch. Moreover, colors, fabrics, overlays and/or the like may be used. The window may also be formed of a thermochromic plastic that is at least partially clear, transparent or translucent so as to allow viewing therethrough but which turns opaque or semi-opaque after installation of the pouch on the user due to body heat of the user. In like manner, the window may be clear, transparent or translucent (clear) but have a thermochromic ink or paint thereon that likewise turns opaque or semi-opaque (opaque) after installation of the pouch on the user due to body heat of the user. In all cases, the thermochromic plastic, ink, paint and the like may have a slight tint or color when clear but which takes on a darker shade or color when heat activated to thereafter become opaque.

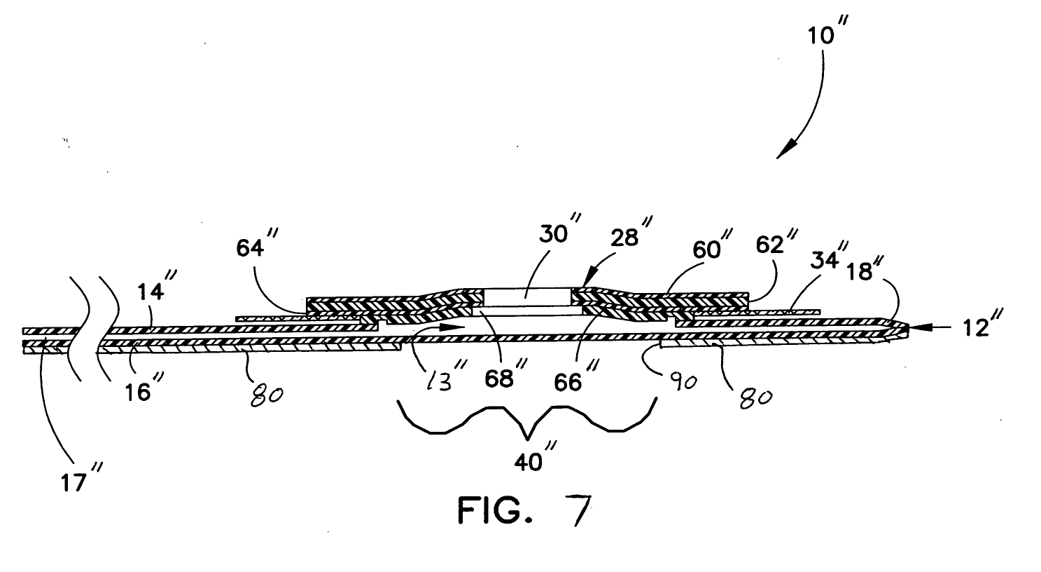

Referring to FIG. 7, there is shown another embodiment of an ostomy pouch generally designated 10″ similar to the ostomy pouch 10 of FIG. 1 and wherein like features are represented by the same reference number with a double prime (i.e. ″). In this embodiment, the back side 16″ is formed of a clear, transparent, translucent, semi-clear, semi-translucent or semi-transparent plastic. The front side 14″ is formed of an opaque, non-transparent, non-translucent or semi-opaque, semi-non-transparent, or semi-non-translucent plastic. In this embodiment, a layer 80 is provided over at least the front side 16″ of the pouch. While not shown, a layer may be provided over the back side 14″. The layer 80 has a viewing window, area or cutout 90 therein the type thereof dependent upon the make-up of the layer 80. The viewing window, area or cutout 90 is positioned to allow the user to see at least a portion of the stoma receptor and/or the stoma opening or orifice during placement of the ostomy pouch on the user. The layer 80 may be an opaque, non-transparent or non-translucent, or semi-opaque, semi-non-transparent or semi-non-translucent plastic, cloth, paper, decal, label, sticker, overlay, lamina, strata/stratum, coat/coating, strata/stratum, deposit, paint, ink, markings or the like. The layer 80 may encompass the back side 14″ if desired. In this manner, the layer 80 is used to obscure, hide, cloak, or otherwise prevent or hinder the viewing of contents of the ostomy pouch except for the window or area 90.

The ostomy pouches shown and/or described herein may also come with a sticker, decal, label, overlay or the like that is configured to be placed over the window once the pouch is in place. This would allow the user to cover the window if desired.

Each one of the present ostomy pouches shown and/or described herein may have the front side, both the front and back sides or the entire pouch formed from a thermochromic plastic, a plastic with thermochromic paint thereon or a plastic with a thermochromic ink thereon (collectively thermochromic material). A thermochromic material is one in which the material change from an initially clear or transparent state in order to allow the user to easily install the ostomy pouch but, which after installation, turns opaque, non-transparent, non-translucent, semi-opaque, semi-non-transparent or semi-non-translucent thereafter due to the user's body heat. The thermochromic material may also start out in a generally clear, transparent or translucent color then turn a darker color that is opaque, non-transparent, non-translucent, semi-opaque, semi-non-transparent or semi-non-translucent due to the user's body heat. The thermochromic paint or ink may be applied as a decal, sticker, label, overly, coat, coating, sheet, film, deposit, strata, stratum, lamina or the like. In another form, the pouch 10 or at least the front side thereof is formed of an opaque plastic or clear plastic with an opaque layer while and the window thereof is formed of the thermochromic material.

In another form, the ostomy pouch or at least a portion thereof is formed from a clear, transparent, translucent, semi-clear, semi-transparent, semi-translucent plastic with a thermochromic material layer, overlay, stratum, coat or coating except for a clear, transparent or translucent viewing window or area positioned to allow the user to see at least a portion of a stoma receptor or opening of the pouch to allow the user to install the ostomy pouch. The window may, however, be formed of a thermochromic material. Various combinations are contemplated and within the scope of the present invention.

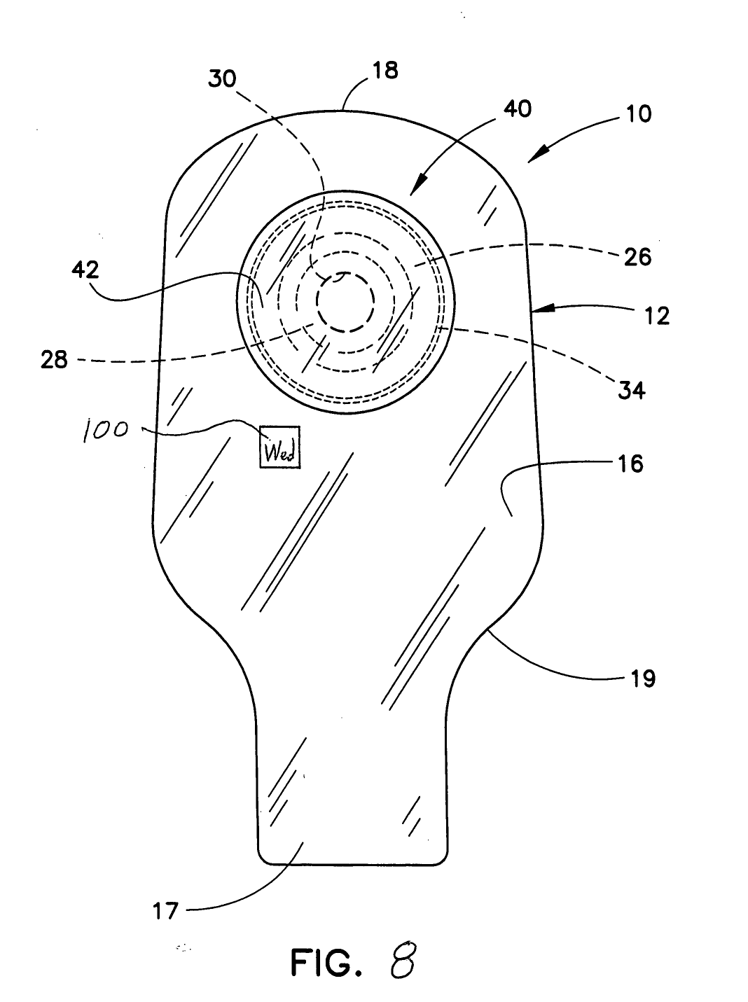

Referring to FIG. 8, there is depicted the ostomy pouch 10 representing all of the embodiments of ostomy pouches or bags shown and/or described herein. In accordance with an aspect of the present invention, the ostomy pouch 10 is configured, adapted and/or operable to have a day of the week applied thereon. The day of the week would be the day that the ostomy pouch has been installed. This provides a reminder to the user as to how long the ostomy pouch has been worn. Application of the day of the week may be provided in a day of the week area 100. Such application may be accomplished by writing the day of the week (shown in FIG. 8 as Wednesday and abbreviated “Wed”) using a writing instrument, or by a day of the week sticker, label, decal or the like. The ostomy pouch 10 could come with a set of days of the week stickers if desired.

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

What is claimed is:

1. An ostomy pouch comprising:
a plastic bag having a front side and a back side and defining an interior for the collection of waste material;
a stoma opening formed in the back side of the plastic bag and in communication with the interior, the stoma opening configured to receive a stoma or a user; and
an opaque layer covering the front side and having a viewing area therein positioned to allow the user to view at least a portion of the stoma opening therethrough.

2. The ostomy pouch of claim 1, wherein the viewing area comprises one of a cutout, a clear plastic and a thermochromic material.

3. The ostomy pouch of claim 1, wherein the opaque layer comprises one of a decal, sticker, label, overly, coat, coating, sheet, film, deposit, strata, stratum, and lamina.

4. The ostomy pouch of claim 1, further comprising an opaque layer covering the back side.

5. An ostomy pouch comprising:
a plastic bag having a front side of an opaque plastic and a back side of a transparent plastic and defining an interior for the collection of waste material;
a stoma opening formed in the back side of the plastic bag and in communication with the interior, the stoma opening configured to receive a stoma or a user; and
a window formed in the opaque plastic of the front side and positioned to allow the user to view at least a portion of the stoma opening therethrough.

6. The ostomy pouch of claim 5, wherein the window is formed of one of a clear plastic and a thermochromic material.

7. The ostomy pouch of claim 5, wherein the window is formed of a clear plastic with a thermochromic layer.

8. An ostomy pouch comprising:
a bag formed of a thermochromic material and defining an interior for the collection of waste material; and
a stoma opening formed in a back side of the bag and in communication with the interior, the stoma opening configured to receive a stoma or a user.
